(19) United States
(12) Patent Application Publication
(10) Pub. No.: US 2009/0264809 A1
(43) Pub. Date: Oct. 22, 2009

(54) METHOD AND APPARATUS OF LOW STRENGH ELECTRIC FIELD NETWORK-MEDIATED DELNERY OF DRUG, GENE, SIRNA, SHRN, PROTEIN, PEPTIDE, ANTIBODY OR OTHER BIOMEDICAL AND THERAPEUTIC MOLECULES AND REAGENTS IN SKIN, SOFT TISSUE, JOINTS AND BONE

(75) Inventor: Luyi Sen, Stevenson Ranch, CA (US)

Correspondence Address: Law Offices of Daniel L. Dawes, 5200 Warner Blvd, Ste. 106, Huntington Beach, CA, 92649, US

(73) Assignee: THE REGENTS OF THE UNIVERSITY OF CALIFORNIA, Oakland, CA (US)

(21) Appl. No.: 12/294,313

(22) PCT Filed: Apr. 2, 2007
(86) PCT No.: PCT/US2007/008445

§ 371 Date: Sep. 24, 2008

Related U.S. Application Data
(60) Provisional application No. 60/744,528, filed on Apr. 10, 2006.
(60) Provisional application No. 60/819,277, filed on Jul. 6, 2006.

(51) Int. Cl. A61N 1/30

(52) U.S. Cl. 604/20

(57) ABSTRACT

The illustrated embodiments of the invention include four preferred embodiments: 1) a method and apparatus for the joint and its related soft tissue for bone gene, protein and drug delivery; 2) a method and apparatus for gene, protein and drug delivery to an extremity; 3) a method and apparatus for delivery of gene, protein and drug delivery to skin and soft tissue; and/or 4) a method and apparatus for delivery of a gene, protein and drug to soft tissue tumor.

### RELATED APPLICATIONS

The present application is related to U.S. Provisional Patent Application Ser. No. 60/744,528, filed on Apr. 10, 2006, and to U.S. Provisional Patent Application Ser. No. 60/819,277, filed on Jul. 6, 2006, which are incorporated herein by reference.

### BACKGROUND OF THE INVENTION

1. Field of the Invention

The invention relates to the field of cellular therapy in skin, soft tissue, joint and bone of large animals and ex vivo and in vivo human of biomedical therapeutic molecules and reagents, including drugs, genes, siRNAs, peptides, proteins, antibodies by means of low strength electric fields.

2. Description of the Prior Art

Electroporation is a technique involving the application of short duration, high intensity electric field pulses to cells or tissue. The electrical stimulus causes cell membrane destabilization and the subsequent formation of nanometer-sized pores. In this permeabilized state, the membrane can allow passage of DNA, enzymes, antibodies and other macromolecules into the cell. Electroporation holds potential not only in gene therapy, but also in other areas such as transdermal drug delivery and enhanced chemotherapy. Since the early 1980s, electroporation has been used as a research tool for introducing DNA, RNA, proteins, other macromolecules, liposomes, latex beads, or whole virus particles into living cells.

Electroporation efficiently introduces foreign genes into living cells, but the use of this technique had been restricted to suspensions of cultured cells only, since the electric pulse are administered in a cuvette type electrodes. Electroporation is commonly used for in vitro gene transfection of cell lines and primary cultures, but limited wok has been reported in tissue. In one study, electroporation-mediated gene transfer was demonstrated in rat brain tumor tissue. Plasmid DNA was injected intra-arterially immediately following electroporation of the tissue. Three days after shock treatment expression of the lac2 gene or the human monocyte chemoattractant protein-1 (MCP-1) gene was detected in electroporated tumor tissue between the two electrodes but not in adjacent tissue.

Electroporation has also been used as a tissue-targeted method of gene delivery in rat liver tissue. This study showed that the transfer of genetic markers β-glactosidase (β-gal) and luciferase resulted in maximal expression at 48 h, with about 30-40% of the electroporated cells expressing bgal, and luciferase activities reaching peak levels of about 2500 pgimg of tissue.

In another study, electroporation of early chicken embryos was compared to two other transfection methods: microparticle bombardment and lipofection. Of the three transfection techniques, electroporation yielded the strongest intensity of gene expression and extended to the largest area of the embryo.

Most recently, a electroporation catheter has been used for delivery heparin to the rabbit arterial wall, and significantly increased the drug delivery efficiency.

Electric pulses with moderate electric field intensity can cause temporary cell membrane permeabilization (cell discharge), which may then lead to rapid genetic transformation and manipulation in wide variety of cell types including bacteria, yeasts, animal and human cells, and so forth. On the other hand, electric pulses with high electric field intensity can cause permanent cell membrane breakdown (cell lysis). According to the knowledge now available, the voltage applied to any tissue must be as high as 100-200 V/cm. If we want use electroporation on a large animal or human organ, such as human heart, it must be several kV. This will cause enormous tissue damage. Therefore, this technique is still not applicable for clinical use.

Electroporation apparatus has been used for skin drug delivery used 2-6 needles to apply high voltage, short duration pulses on the skin. This system caused significant skin damage and inflammation due to the needle direct injury and the high voltage shock that limited its use. The patent of a microchip device published recently for skin electroporation that will also use high voltage although it has not been used in human animal yet.

### BRIEF SUMMARY OF THE INVENTION

A plurality of embodiments are disclosed and enabled illustrating how to apply LSEN or low voltage pulses to tissue with acceptable transfection efficiency for gene, protein and drug delivery systems. The first is a method and apparatus for joint and its related soft tissue and bone gene, protein and drug delivery. In this system, a long injection needle with a catheter is inserted into the joint sac, then the guiding needle was taken out. A drug, gene, siRNA, shRNA, peptide, protein, antibody or any other biomedical therapeutic reagent, or a combination thereof is injected into the catheter. In addition, an inhibitor, enhancer, agonist, antagonist, regulator, modulator, modifier, or monitor, or any combination thereof of the drug, gene, siRNA, shRNA, peptide, protein, antibody or a biomedical therapeutic molecule or reagent may be employed. Then, the joint is mobilized, letting the gene uniformly distributed in the joint. Then the wire with a positive electrode on the tip of the wire is inserted into the catheter. The tip of the wire extends out of the catheter. Then a pad with an array of the negative electrodes are used cover the whole joint. All negative electrodes are placed into tight contact with the skin of the joint with conducting gels and folding clips and bands. Then, a low strength electric field network is applied.

The second embodiment is a method and apparatus for gene, protein and drug delivery to an extremity. In this embodiment, there are three different ways to deliver drug, gene, siRNA, shRNA, peptide, protein, antibody or any other biomedical therapeutic reagents into the extremities. First, there is intravesculary (venous and arterial), gene, siRNA, shRNA, peptide, protein, antibody or any other biomedical therapeutic reagents delivery using a iv pump or other controller. The delivery should be continuous during the application of electric field. Second, the gene, siRNA, shRNA, peptide, protein, antibody or any other biomedical therapeutic reagents can be applied topically with solution, oil, gel or other drug delivery materials. Third gene, siRNA, shRNA, peptide, protein, antibody or any other biomedical therapeutic reagents can be applied by subcutaneous injection. The array of positive and negative electrodes are applied in the same or similar manner as with an extremity and the limbs.

The low the low strength electric field network LSEN is applied. The array of the electrodes can be made on a glove for the hand, a sock for the foot, or a sleeve for arm, or other means for conforming to the body or tissue surface to insure all electrodes are tightly contacted on the skin.

The third embodiment is a method and apparatus for gene, protein and drug delivery to the body surface (including skin and soft tissue). In this embodiment, the methods for delivery drug, gene, siRNA, shRNA, peptide, protein, antibody or any other biomedical therapeutic reagents are the same as that for the extremity and limbs. The topical application is believed to be more practical. The array of positive and negative electrodes are applied on the body surface in the same or similar manner as describe above using tape, gel or bandages to fix the electrode array.

The fourth embodiment is a method and apparatus for soft tissue tumor gene, protein and drug delivery. In this embodiment, the methods for delivery drug, gene, siRNA, shRNA, peptide, protein, antibody or any other biomedical therapeutic reagents will be the same or similar to that for extremity and limbs. A local injection can be used for tumors. The array of positive and negative electrodes as applied to the body surface can be used if the tumor is superficial. Alternatively, the negative electrodes array are applied on one side and the positive electrodes on the another side of the tumor if the tumor is on the extremity or limb. Thus, the fringing electric fields can passing through the tumor using adhesion material, tapes, gel or bandage to fix the electrode array. If intravascular delivery is applied, the drug, gene, siRNA, shRNA, peptide, protein, antibody or any other biomedical therapeutic reagents delivery should be performed during the application of LSEN to the target tissue.

In one embodiment of the invention use is made of a dense electrode array and a central internal electrode to generate the electrode field fringe network that through the whole joint. A more dense electrode array generates a more uniformed electric field fringe network distributed throughout the whole joint. The joint cavity is a closed chamber. The gene or drug injected into the joint cavity will remain in place for a long time. After the gene and/or drug is injected into the joint, the joint is moved to help the drug and/or gene to be distributed to whole joint cavity.

An internal electrode wire is inserted into the joint though the same catheter that be used for inject gene or drug. The catheter is pulled out from the joint and the tip of the wire should be placed in the center of the joint. The whole wire is insulated, except for the small tip which is plated with a highly conductive material, such as platinum. Thus, when a power gradient or voltage is applied on the exterior electrodes of array and internal electrode wire, the electric field fringes can across through all of structures of the joint, that include bone, cartilage, ligaments, tendons, muscle and soft tissues. This is the most efficient way of utilizing the electric energy of the electric field, because the all electric fringes can be used for a driving force for the drug or gene delivery.

For intracellular delivery of a positively charged molecule, electrodes on array on the body surface should be connected to the negative pole of the pulse generator. The positive molecules will travel follow the electric fringes from the joint cavity toward the body surface. For intracellular delivery of a negatively charged molecule, electrodes of array on the body surface should be connected to the positive pole of the pulse generator. Thus, negative molecules will also travel follow the electric fringes from the joint cavity toward the body surface.

This device and method can be used for any joint application, such as knee, shoulder, wrist, elbow, ankle, finger, hip, etc. If it is not be able to wrap the whole joint, a flat circuit can be used, such as spinal joint, jaw or the like. FIGS. 4a-4e illustrate the range of applications to which the unipolar electrode of the invention may be used, showing by way of example only unipolar applications to a knee joint, a shoulder joint, an elbow joint, a wrist joint and tendons, and an ankle joint. In each case an internal electrode is inserted and the joint is wrapped in a unipolar array which closely or intimately conforms to the exterior shape of the joint.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1a is a top plan view of a unipolar array devised according to the invention used to create an LSEN field which is used to drive genes or drugs into tissue.

FIG. 1b is a side cross-sectional view of FIG. 1a as seen through lines 1b-1b.

FIG. 2a is a diagram of a first step in a method illustrating the method of the invention wherein a knee joint cavity is treated according to the invention by insertion of a catheter and a gene or drug into the joint cavity.

FIG. 2b is a diagram of a second step in the method of FIG. 2a where an electrode wire is inserted into the joint and the gene and/or drug distributed in the joint cavity by movement of the joint.

FIG. 2c is a diagram of a third step in the method of FIGS. 2a and 2b where an electrode array is disposed around the joint and the gene and/or drug driven into the tissue by an LSEN field applied to the joint cavity.

FIG. 2d is a waveform diagram illustrating the general form of the LSEN field protocol applied in the method of FIGS. 2a-2c.

FIG. 3a is a top plan view of a bipolar array devised according to the invention used to create an LSEN field which is used to drive genes or drugs into tissue.

FIG. 3b is a side cross-sectional view of FIG. 3a as seen through lines 3b-3b.

FIG. 3c is a side cross-sectional view of a second embodiment FIG. 3a as seen through lines 3b-3b where a drug eluting pad is added to the array.

FIG. 4a is a depiction of application of the invention to a knee joint.

FIG. 4b is a depiction of application of the invention to a shoulder joint.

FIG. 4c is a depiction of application of the invention to an elbow joint.

FIG. 4d is a depiction of application of the invention to a wrist joint and tendons.

FIG. 4e is a depiction of application of the invention to an ankle joint.

FIG. 5a is a top plan view of a bipolar body surface electrode array in combination with a drug eluting system.

FIG. 5b is a top plan view of a bipolar body surface electrode array in combination with a drug seepage system.

FIG. 5c is a side cross sectional view of a bipolar body surface electrode array in combination with a drug seepage system as seen through section lines 5c-5c in FIG. 5b.

FIG. 6a is a photographic depiction of the drug delivery system of the invention as applied to body skin.

FIG. 6a-Step I and Step II are photographic depictions of the drug delivery system of the invention as applied to body skin.

FIG. 6b-Step I and Step II are photographic depictions of the drug delivery system of the invention as applied to the scalp.

FIG. 6c-Step I and -Step II are photographic depictions of the drug delivery system of the invention as applied to a limb extremity.

FIG. 6d is a perspective illustration of the drug delivery system of the invention as applied to skin showing the dermal structures in relation to the array.

FIG. 7a-Step I and -Step II are depictions of the drug delivery system of the invention as applied to gene infusion into a hand.

FIG. 7b is a depiction of the drug delivery system of the invention as applied to gene infusion into a foot.

FIG. 8a is a microphotograph of showing in situ hybridization of transgene expression in articular cartilage of a knee in the embodiment of IL-10 gene transfer using the invention.

FIG. 8b is a microphotograph of showing in situ hybridization of transgene expression in articular cartilage of a knee in the embodiment of liposome-mediated IL-10 gene transfer using the invention.

FIG. 8c is a graph showing the efficiency of gene transfer in the percentage of positive stained cells for in situ hybridization in which the invention, liposome mediated and plasmid mediation are compared.

FIG. 8d is a graph showing the transgene expression level determined by quantitative reverse transcription-polymerase chain reaction (qRT-PCR) comparing use of the invention with liposome mediation.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment of the invention is a methodology and an apparatus for performing a method for facilitating the targeting of drug, gene, siRNA, shRNA, peptide, protein, antibody or any other biomedical therapeutic molecules and reagents into the cells of skin, soft tissue, joint and bone of large animal and/or humans in ex vivo and in vivo contexts as assisted with the application of a low strength electric field network. Drug, gene, siRNA, shRNA, peptide, protein, antibody or biomedical therapeutic molecules and reagents, include by way of example genes, proteins and antibodies thereof for:
1) leukocyte markers, such as CD2, CD3, CD4, CD5, CD6, CD7, CD8, CD11a,b,c, CD13, CD14, CD18, CD19, CD20, CD22, CD23, CD25, CD27 and its ligand, CD28 and its ligands B7.1, B7.2, B7.3, CD29 and its ligand, CD30 and its ligand, CD40 and its ligand gp39, CD44, CD45 and isoforms, Cdw52 (Campath antigen), CD56, CD58, CD69, CD72, CD80, CD86, CTLA-4, CTLA4Ig, LFA-1 and TCR. or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent.
2) histocompatibility antigens, such as MHC class I or II, the Lewis Y antigens, Slex, Sley, Slea, and Selb;
3) adhesion molecules, including the integrins, such as VLA-1, VLA-2, VLA-3, VLA-4, VLA-5, VLA-6, LFA-1, Mac-1, αVβ3, and p150, 95; and
4) the selectins, such as L-selectin, E-selectin, and P-selectin and their counterreceptors VCAM-1, ICAM-1, ICAM-2, and LFA-3;
5) interleukins, such as IL-1, IL-2, IL-3, IL-4, IL-5, IL-6, IL-7, IL-8, IL-9, IL-10, IL-11, IL-12, IL-13, IL-14, and IL-15;
6) interleukin receptors, such as IL-1R, IL-2R, IL-3R, IL-4R, IL-5R, IL-6R, IL-7R, IL-8R, IL-9R, IL-10R, IL-11R, IL-12R, IL-13R, IL-14R and IL-15R;
7) chemokines, such as PF4, RANTES, MIP1a, MCP1, IP-10, ENA-78, NAP-2, Gro-α, Gro-β, and IL-8;
8) growth factors, such as TNFα, TGFβ, TSH, VEGF/VPF, PTHrP, EGF family, FGF, PDGF family, endothelin, Fibrosin (F.sub.sF.sub.−1), Laminin, and gastrin releasing peptide (GRP);
9) growth factor receptors, such as TNFαR, RGFβR, TSHR, VEGFR/VPFR, FGFR, EGFR, PTHrPR, PDGFR family, EPO-R, GCSF-R and other hematopoietic receptors;
10) interferon receptors, such as IFN-aR, IFN-βR, and IFN.sub.YR;
11) Igs and their receptors, such as IGE, FceRI, and FceRII;
12) tumor antigens, such as her2-neu, mucin, CEA and endosialin;
13) allergens, such as house dust mite antigen, IoI p1 (grass) antigens, and urushiol;
14) viral proteins, such as CMV glycoproteins B, H, and gCIII, HIV-1 envelope glycoproteins, RSV envelope glycoproteins, HSV envelope glycoproteins, EBV envelope glycoproteins, VZV, envelope glycoproteins, HPV envelope glycoproteins, Hepatitis family surface antigens;
15) toxins, such as pseudomonas endotoxin and osteopontin/uropontin, snake venom, spider venom, and bee venom;
16) blood factors, such as complement C3b, complement C5a, complement C5b-9, Rh factor, fibrinogen, fibrin, and myelin associated growth inhibitor;
17) enzymes, such as cholesterol ester transfer protein, membrane bound matrix metalloproteases, and glutamic acid decarboxylase (GAD); and
18) miscellaneous antigens including ganglioside GD3, ganglioside GM2, LMP1, LMP2, eosinophil major basic protein, PTHrp, eosinophil cationic protein, pANCA, Amadori protein, Type IV collagen, glycated lipids, nu-interferon, A7, P-glycoprotein and Fas (AFO-1) and oxidized-LDL;
19) calcineurin inhibitor, e.g. cyclosporin A or FK 506;
20) mTOR inhibitor, e.g. rapamycin, 40-O-(2-hydroxyethyl)-rapamycin, CCI779, ABT578 or AP23573;
21) an ascomycin having immunosuppressive properties, e.g. ABT-281, ASM981, etc.;
22) corticosteroids; cyclophosphamide; azathioprene; methotrexate; leflunomide; mizoribine; mycophenolic acid; mycophenolate mofetil; 15-deoxyspergualine or an immunosuppressive homologue, analogue or derivative thereof; and
23) apoptosis genes or
24) any combination of the members of the above group.

The compounds may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g. a malignant cell anti-proliferative agent. By the term “chemotherapeutic agent” is meant any chemotherapeutic agent and it includes but is not limited to:
i. an aromatase inhibitor,
ii. a microtubule active agent, an alkylating agent, an antineoplastic antimetabolite or a platin compound,
iii. a compound targeting/decreasing a protein or lipid kinase activity or a protein or lipid phosphatase activity, a further anti-angiogenic compound or a compound which induces cell differentiation processes,
iv. a bradykinin 1 receptor or an angiotensin II antagonist,
v. a cyclooxygenase inhibitor, a bisphosphonate, a histone deacetylase inhibitor, a heparanase inhibitor (prevents heparan sulphate degradation), e.g. PI-88, a biological response modifier, preferably a lymphokine or interferons, e.g. interferon .quadrature., an ubiquitination inhibitor, or an inhibitor which blocks anti-apoptotic pathways,
vi. an inhibitor of Ras oncogenic isoforms, e.g. H-Ras, K-Ras or N-Ras, or a farnesyl transferase inhibitor, e.g. L-744,832 or DK8G557,
vii. a telomerase inhibitor, e.g. telomestatin,
viii. a protease inhibitor, a matrix metalloproteinase inhibitor, a methionine aminopeptidase inhibitor, e.g. bengamide or a derivative thereof, or a proteosome inhibitor, e.g. PS-341, and/or
ix. a mTOR inhibitor, or
x. any combination of members of the group.

A low strength electric field network system is used for transferring any therapeutic gene, siRNA, shRNA, protein or drug into the isolated limb, joint, skin and tissue ex vivo, or extremity, joint or body surface in vivo, such as soft tissue, muscle, tendon, bone, or cartilage. This invention has been tested on the rabbit joint and skin.

The illustrated embodiments of the invention include four preferred embodiments: 1) a method and apparatus for the joint and its related soft tissue for bone gene, protein and drug delivery; 2) a method and apparatus for gene, protein and drug delivery to an extremity; 3) a method and apparatus for delivery of gene, protein and drug delivery to skin and soft tissue; and/or 4) a method and apparatus for delivery of a gene, protein and drug to soft tissue tumor.

The illustrated embodiment addresses the shortcomings of the prior art by providing a low strength electroporation-mediated gene, protein and drug delivery in the isolated organs and tissue ex vivo, and in vessels and tissue in vivo. For proofing of the concept, we conducted a series studies using the low strength electroporation system of the invention for gene delivery in large animal hearts ex vivo and in vivo. We found this method has highest gene transfer efficiency and efficacy, and that it is higher than any existing viral and nonviral gene transfer techniques. We did not find any cardiac and adverse effect in large animals to date. Further, the low strength electroporation system of the invention has been specifically extended for application to the skin, soft tissue, joint and bone gene, protein, and drug delivery.

The illustrated embodiment of the invention is a strategy for electro-permeabilization of the cell membrane for gene, protein, drug targeting in skin, soft tissue and bone ex vivo and in vivo using an array of electrodes forming a network to apply the electric field with low voltage, short pulse duration, burst pulses for a long period time. The nature of the electromagnetic field pattern provided by the network is so different than convention the nature of the electromagnetic field pattern provided by conventional electroporation, that the for the purposes of this specification, the field itself is referenced not as an electroporation field, but as a low strength electric field network (LSEN).

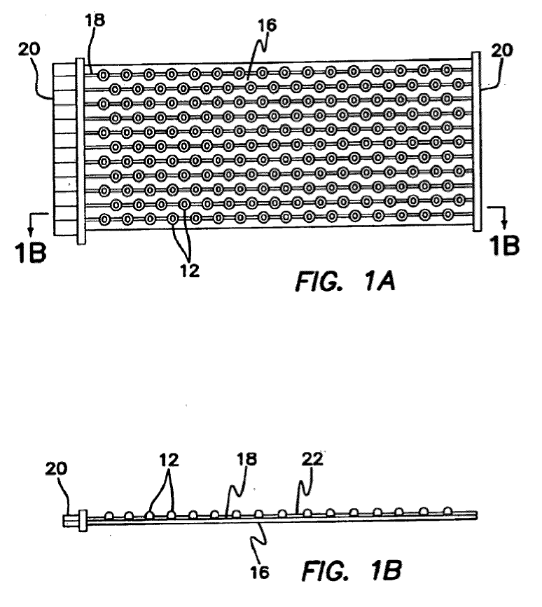

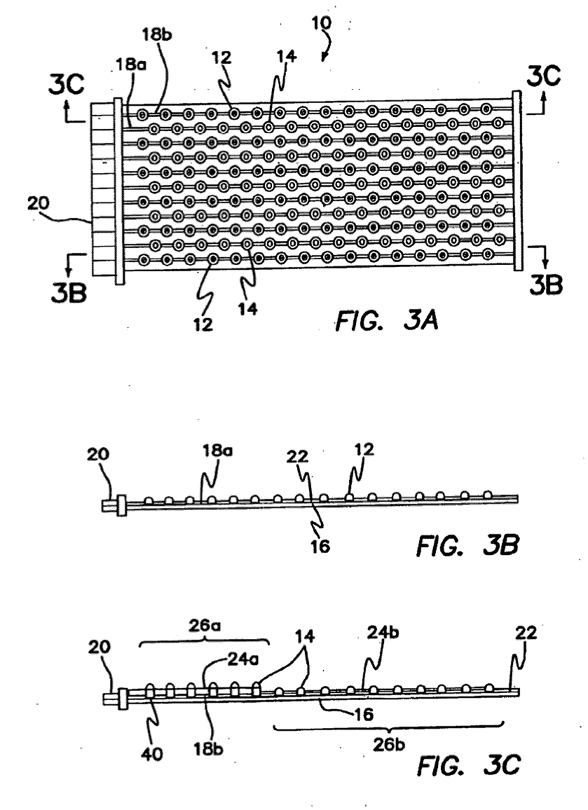

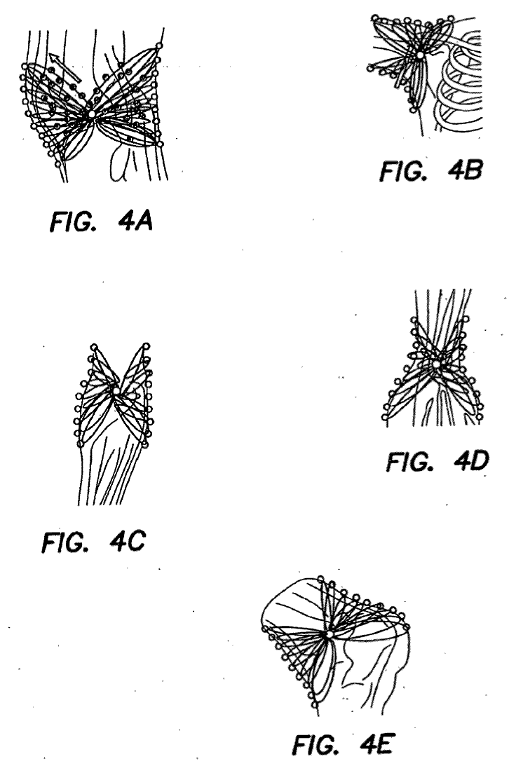

FIG. 1a is a plan top view of a unipolar body surface electrode array and FIG. 3a is plan top view of a bipolar body surface electrode array usable in the invention. However, it must be understood that the arrays which may be provided and effective as sources of LSEN are not limited to these two examples, but include any arrays now known or later devised which perform the same or similar functions.

The arrays of FIGS. 1a and 3a comprise a flexible electrode array 10. A plurality of electrodes 12, 14 are coupled to either a positive voltage source (not shown) or negative voltage source or pulse generator (not shown) respectively. The cylindrical electrodes 12, 14 are mounted or carried on a flexible substrate or adhesion pad 16 and aligned in rows by connection or coupling to a plurality of conductive lines or wires 18. Wires 18 are coupled at their opposing ends to a multiple pin connector 20. In the unipolar embodiment of FIG. 1a each wire 18 is provided with the same polarity voltage. In the bipolar embodiment of FIG. 3a every other wire 18 is provided with a voltage of opposite polarity. Wires 18 in the embodiment of FIG. 1a and wires 18a and 18b in the embodiment of FIG. 3a may be insulated, but electrically coupled to each electrode 12, 14 in its row. For example in the bipolar embodiment of FIG. 3a wire 18a is coupled to a row of electrodes 12 of one voltage polarity and wire 18b to a row of electrodes 14 of the opposite voltage polarity. As shown in FIGS. 1a and 3a electrodes 12, 14 in adjacent rows are offset from each other in other to increase electrode density on pad 16. The electrodes 12, 14 are shaped cylinders with an average diameter is preferably equal to or smaller than 2 mm. The electrode surface extends prominently from the plane of array 10 by at least 0.05 cm. Wires 18 are preferably approximately 0.5 cm apart and electrodes 12 are placed along each wire 18 with a 0.3 cm spacing from the surface of one electrode 12 to the surface of the next adjacent one connected to the same wire 18. The diameter of electrode 12, 14 is approximately 0.15 cm. Thus, the projecting electrodes 12, 14 can tightly contact the body surface skin. All electrodes are preferably plated with platinum or other conductive biocompatible material. The entire array 10 is preferably covered by an insulation layer 22. Only a very small area, the tip of the spherical, <0.05 cm² of electrode 12, 14 is directly contacted on the skin. Thus, the chance of the heat damage will be reduced to the minimal. The size of the various elements of the array 10 depend on its application and those provided here are only for illustration. The shape of the array 10 will also change depending on the nature of its end application. Shape and size changes can be made according to the teachings of the invention with the additional use of ordinary design principles.

FIG. 1b is a side cross-sectional view as seen through lines 1b-1b of the plan view of FIG. 1a. Unipolar electrodes 12 are shown as being bullet shaped cylinders of approximately 0.075 cm height contacting wire 18 at the base of the cylinder, which wire 18 is carried on pad 16, and which cylinders have a blunt nose extending through insulation layer 22 for contact with the skin or tissue. It is expressly understood that the contact surface or nose of electrodes 12, 14 may be varied to assume any desired shape including more flattened, pointed, conical or needle-like terminations.

Similarly FIGS. 3b and 3c show a side cross-sectional view of two embodiments of a bipolar array 10 as seen through lines 3b-3b of the plan view of FIG. 3a. The side cross-sectional view of FIG. 3b may be either polarity electrode 12 or 14 coupled to wires 18a or 18b respectively. The configuration of FIG. 3b is identical to that described in connection with FIG. 1b, while the embodiment of FIG. 3c shows a first group 26a of electrodes 14 provided with an increased cylinder height, while a second group 26b has the original or same cylinder height of electrodes 12 of FIG. 3b, namely 0.075 cm. The height of electrodes 12, 14 are increased in group 26a by means of an insulated cylindrical shim 40. A drug eluting pad 24 is disposed on layer 22, but not covering, group 26a of electrodes 14. Drug eluting pad 24 is electrically insulated from electrodes 12, 14 by means of the insulating coating or layer on shim 40. The use of the drug eluting pad 24 will be described in greater detail below. In addition to height differences the electrodes 14 of group 26a and group 26b may be differentiated from each other ways, such as shape, material composition, structure and any other design parameter desired. Pad 24 is shown as selectively disposed on array 10, but it is also contemplated that the entire array 10 or multiple selected portions of array 10 may be provided with pad 24.

The embodiment of FIGS. 3a-3c is intended for the drug delivery applications for superficial areas and/or in applications where there is no way to insert a internal central electrode, such as in the case of delivery to skin, subcutaneous tissue, soft tissue, scalper, face, torso, hand, foot, and the like.

Although the main structures of the embodiment of FIGS. 1a-1b and FIGS. 3a-3c are the same, there are several differences. Since there is no internal electrode, both positive and negative electrodes 12, 14 are included on the same array 10. Wires 18a and 18b providing lines of negative electrodes and positive electrodes or vice versa are alternatively arranged on the same pattern as shown in the plan view of FIG. 1a of array 10. For the application to small area, such as for wound healing, for hair follicles in trichomadsis, skin lesion and scare or wrinkle remove etc, the density of electrodes 12, 14 will be increased and the overall size of electrodes 12, 14 should be reduced along with the reduced of the size of array 10.

For the small array 10, tape fixed around the array 10 can be used to fix array 10 onto the skin. Additional tape and bandage added on array can insure a tight contact between electrodes 12, 14 and skin. An ointment, oil, fluid, gel, powder or other formula containing the gene and drug can be directly applied on the skin before fixing the array 10 to the skin. Drugs also can be applied by direct injection into the skin using single or multiple injections or by injection or infusion intravascularly.

Wires 18 are made with copper or other conductive material. Preferably, wires 18 are mounted on or in pad 16, which is made from a biocompatible material, such as plastic membrane or other material that is very flexible and which can be tensioned, molded or shaped to make all electrodes 12, 14 tightly contact on the adjacent skin or tissue. Using tape, a bandage, or an air bag (not shown) on array 10 can further compress pad 16 on the skin or tissue to increase the degree of direct contact of electrodes 12, 14 and the skin or tissue. The more tight the contact between electrodes 12, 14 and skin or tissue, the better the conductance, and also the less the electrical heat damage.

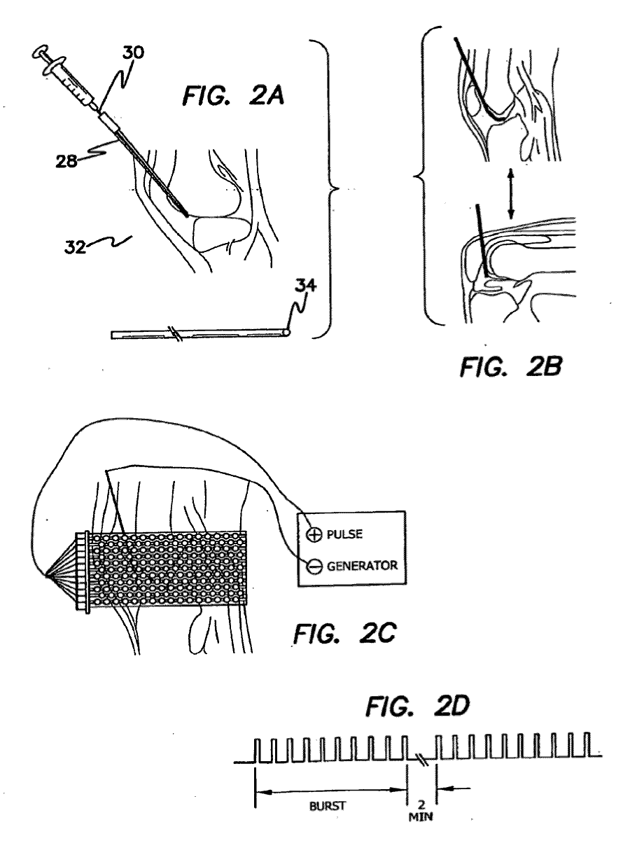

FIGS. 2a-2c use the knee as the example of the method of the illustrated embodiment of the invention. The first step is to insert a vascular catheter 28 with the needle 30 into the knee joint cavity 32, then take the needle 30 out. Inject the biomedical agents or drug, then insert an internal electrode 34 into the catheter 28. The catheter tip should be advanced to the center of the joint cavity 32. An electrode wire is then inserted into the catheter. The internal electrode 34 can be made with copper, stainless steel, or other biocompatible materials, and covered by the insulation layer. Only the exposed tip of the wire is plated with platinum. The tip should be very small, <1 mm³. The wire should be made to very flexible and soft, and to be able to avoid any tissue damage during insertion. Then the catheter 28 can be pulled out from the joint. Then move the joint to let the gene or drug to evenly distribute in the joint cavity 32 as depicted in FIG. 2b.

Then, we can wrap the whole joint with the unipolar body surface electrode array 10 of FIGS. 1a-1b as shown in FIG. 2c. All electrodes 12 will be tightly contacted on the skin using a bandage, tape or a pressure bag. For intracellular delivery of a negatively charged molecule, electrodes on the array 10 should be connected to the positive pole of the pulse generator 36 as shown in FIG. 2c. For intracellular delivery or a positively charged molecule, electrodes 12 on the array 10 should be connected to the negative pole of the pulse generator 36. For a neutral molecule, polarity of connection can be used. Then, LSEN burst-pulses are applied.

The LSEN burst-pulse protocol as depicted by the waveform diagram of FIG. 2d is comprised of approximately 5-50 short duration pulses each with an approximate 2-20 msec pulse duration separated by an approximate 5-30 msec pulse interval in bursts separated by an approximate 1-5 min interburst interval. The strength of the electric field is approximately 0.1-50 volt/cm. The total therapeutic burst sequence can be from 1 sec to several hours.

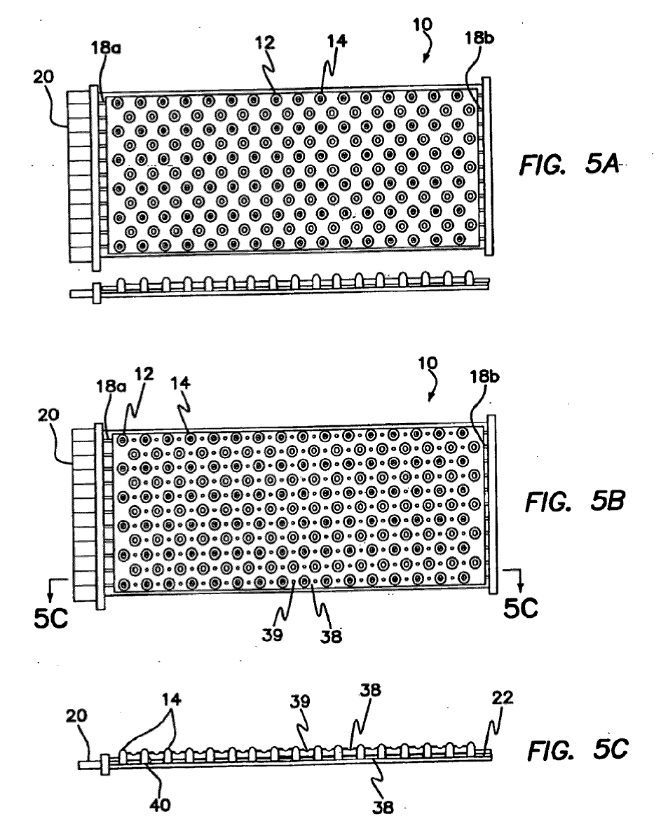

In FIGS. 3c and 5a, a bipolar array 10 is combined with the slow drug release or drug eluting pad 24a to form a complete body surface LSEN-drug delivery system. The slow drug release or drug eluting pad 24a need be provided across the entire array 10, nor provided to the same degree. A portion of pad 24b is thinner and includes therefore a lower cumulative dosage or no dosage of the drug and can be provided a selected portion of array 10. The main structures are the same as that described in the bipolar electrode array device of FIGS. 3a-3b. In addition a slow drug-releasing pad 24a is added on the top of the insulation layer 22. In order to not let the drug releasing pad 24a cover the electrodes 12, 14, the holes are made in the pad 24a to let electrodes 12, 14 pass through the pad 24a. All electrodes 12, 14 are made longer by adding a shim 40 that will accommodate the thickness of the pad 24a. The material of the shim 40 of the electrode 12, 14 is the same as the electrode itself, but with an insulation layer isolating the shim 40 and the pad 24a. Only the tip of the electrode is plated with highly conductive material, such as platinum.

To be successfully used in controlled slow drug releasing formulations, the material of pad 24a must be chemically inert and free of leachable impurities. It must also have an appropriate physical structure, with minimal undesired aging, and be readily processable. Some of the materials that are currently being used or studied for controlled drug delivery include: poly(2-hydroxy ethyl methacrylate); poly(n-vinyl pyrrolidone),poly(methyl methacrylate), poly(vinyl alcohol), poly(acrylic acid), polyacrylamide, poly(ethylene-co-vinyl acetate), poly(ethylene glycol), poly(methacrylic acid). However, in recent years additional polymers designed primarily for medical applications have entered the arena of controlled release. Many of these materials are designed to degrade within the body, among them are: polylactides (pla), polyglycolides (pga), poly(lactide-co-glycolides) (plga), polyanhydrides, polyorthoesters. Those materials can be used as well.

Pad 24a may be replaced by a slow drug release bag 38 as shown in FIGS. 5b and 5c. Slow drug release bag 38 is used as a drug reservoir to form a complete body surface LSEN-drug delivery system. Microholes 39 made in the bag 38 slowly release the drug on to the body surface. The speed of the drug release can be controlled by a compression force applied to the bag. This system is more suitable for delivering the fluid and thin oil or gel formulation. An air bag or tape (not shown) can be added for a driving force for drug release from the slow release bag 38. This embodiment is advantageously used on the extremity or torso. For flat body surface, an infusion tub set and a fluid control pump can be used for controlling the drug into and out from the bag 38, and then for control the drug release from the bag 38.

There is no need for add the insulation layer on the shim of the electrode 12, 14, since the plastic bag is not conductive. The shim 40 still needs to be added under each electrode 12, 14 to raise the electrode 12, 14 so that it can make tight contact with the body surface.

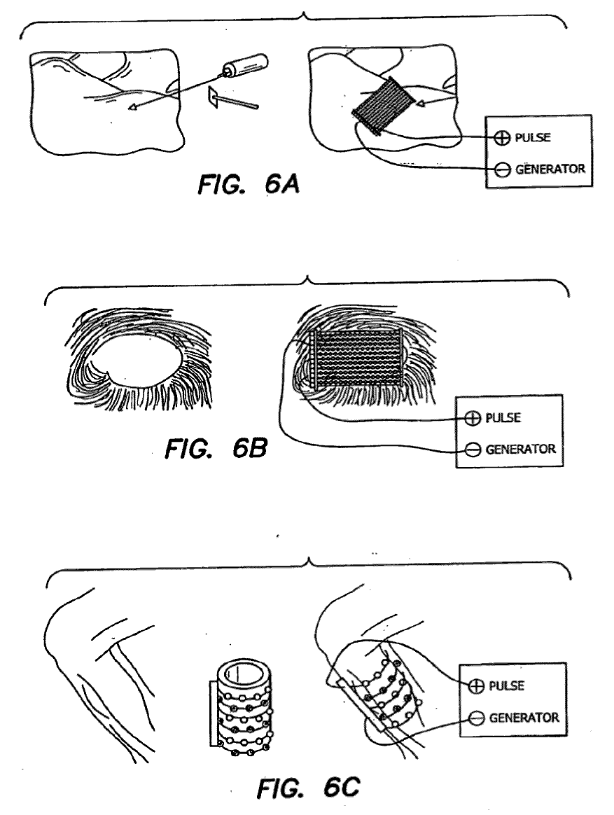

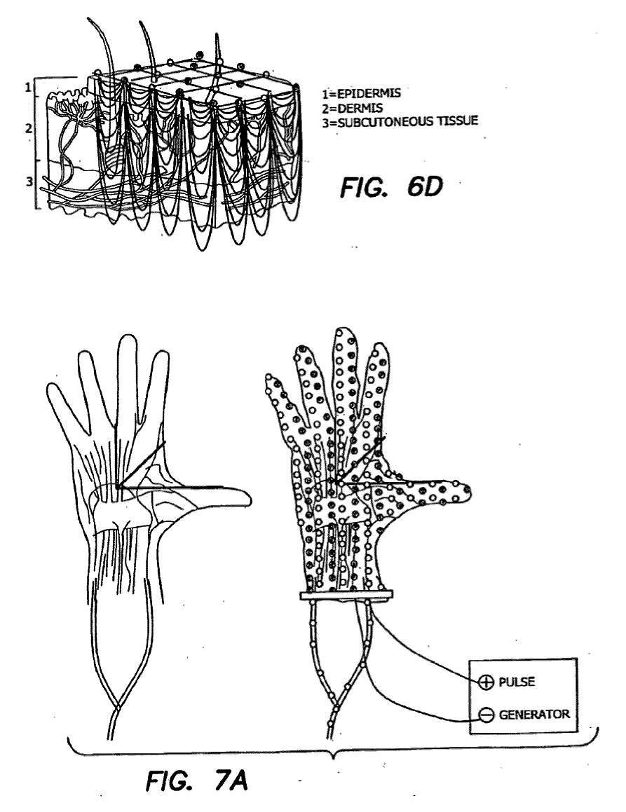

The method of using positive and negative electrodes in an alternative pattern as shown in FIGS. 3a-3c generates an electric field that is parallel with the body surface. The electric field fringes pass through the skin, subcutaneous tissue and deeper structures parallel with the plan of the skin in the network field pattern. In this case, the more distance there is between the skin and electrodes 12, 14, the less electric field strength is seen by the deeper tissues. Therefore, a bipolar array 10 is better be used for the superficial tissue gene and drug delivery as shown in the embodiments of FIGS. 6a-6d.

On another hand, increasing the density of electrodes 12, 14 makes the distance between the positive and negative pairs of the electrodes shorter for a given amount of applied voltage. The strength of the electric field is the volt/cm of the distance between the pair of negative and positive electrodes. Thus, the strength of the electric field in the tissue more distant from electrodes 12, 14, will be increased. In another words, as the electric force between two electrodes is reduced, the strength of the electric field increases vertically in the tissue structures of the skin as depicted in the illustration of FIG. 6d. Thus, even the structures in deep area, such as soft tissue, adipose tissue, muscle, small vessels, nerves, tendon, bone, cartilage can also be reached using a system with increased electrode density. In addition, a more dense electrode pattern will make the electric field network pattern more uniformly distributed in the skin and tissue.

One embodiment of the invention is a method of LSEN-drug delivery in skin wound using a bipolar array 10 in which the gene and drug are applied topically, such as to the chest as in FIG. 6a-step I and -step II. The drug can be fluid, gel, ointment, powder, or other formula. After the drug is applied on the body surface using a dispenser to dispense the drug evenly in the area of application as shown in FIG. 6a-step I, the bipolar array 10 is then applied to the area and connected to the pulse generator 36 as shown in FIG. 6a-step II. Electric pulse can be applied for seconds to hours as described above.

In another embodiment the LSEN-drug delivery in the scalp is performed using a bipolar array 10 with a drug slow release pad 24 as shown in the treatment of FIG. 6b-step I and step II. The trichomadesis area can be covered with the bipolar array 10 combined with a drug slow release pad 24 as shown in FIG. 6b-step I. After the array 10 is connected to the positive and negative poles of the pulse generator 36, the electric pulses are applied as described above as shown in the treatment of FIG. 6b-step II. Drug can also be applied by multiple injection or topical formulas as described above.

In yet another embodiment of LSEN-drug delivery in extremity or torso using bipolar body surface electrode array with the drug slow release bag in a manner similar to the use of pad 24 described above as also shown in FIG. 6c-step I. A pressurized air bag or bandage can be used for controlling the force on the drug release bag. An infusion or injection tub set with a pump is the preferred way to control the drug release in approach as the LSEN field is applied as shown in FIG. 6c-step II. In an extremity, such as diabetes leg, drug can also be delivered vascularly into the vessels. The bipolar array 10 is then used to assist the drug delivery.

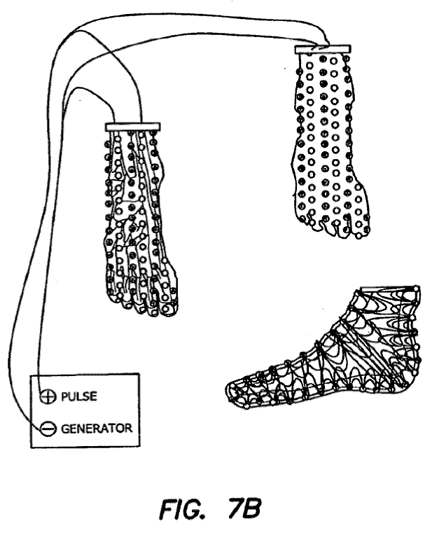

In summary, it must be understood that the disclosed method and apparatus for gene, protein and drug delivery to a joint and its related soft tissue and bone is used in the treatment of any joint diseases and/or joint related bone, cartilage, ligaments, and muscle diseases. The disclosed method and apparatus is used for gene, protein and drug delivery to an extremity for treatment of any diseases in hand and foot, such as primary Raynaud's disease and secondary Raynaud's syndrome, diabetes foot syndrome, Burgers syndrome, rheumatoid arthritis, or similar diseases or conditions as illustrated in FIGS. 7a and 7b where gene infusion is implemented through local vascular injection or topical application by a topical gel or by a drug eluting pad fit into the sock- or glove-defining array 10. This embodiment can also be used for any diseases in limbs, such as varix, varicose ulcer, thrombosis, any embolisms, soft tissue tumors, long bone tumors, and any soft tissue diseases. The disclosed method and apparatus for delivery of genes, proteins and drugs to a body surface including skin and soft tissue is used for skin and soft tissue diseases, superficial soft tissue tumors on the body, such as any kind of wound (surgical wound, scar, burns, etc), skin diseases, skin cancer, skin ulcers, trichomadesis, vitiligo, skin care (remove wrinkles, etc.), any tumors, sarcoma on the body. This embodiment also can be used for ex vivo delivery of immunosupressive agents and anti-inflammation agents to the donor skin, soft tissue, bone or joint for transplantation. The method and apparatus for gene, protein and drug delivery to soft tissue tumors is used for tumors located relatively deeper in the limbs, or extremities, such as sarcoma, bone tumor.

This invention opens a new era for the gene, protein and drug targeting in skin, soft tissue, joint and bone of large animal and human prevention and treatment of large animal and human disease in vivo and ex vivo. There is no existing technique which is applicable for use in humans.

The illustrated embodiments of the invention have four major advantages: 1) the low voltage used reduces the cell damage; 2) more pulses and longer time can be applied to increase the gene and drug delivery efficiency; 3) more even distribution and homogenous strength of electrical field can be applied on the tissue surface by using an electric field network; 4) better electrode-to-skin contact saves energy and significantly reduces skin damage.

As a proof of concept, we conducted an experiment to use the LSEN unipolar electrode array 10 for the gene delivery in rabbit knee. Its method has been described in the above. Briefly, under general anesthesia, a catheter with needle was inserted into the rabbit knee. The needle was then pulled out. About 50 μl joint fluid was draw into the syringe and discarded, then 100 μl of plasmid IL-10 gene (100 μg) was injected into the knee. An internal electrode wire was inserted into the catheter and position in the center of the knee. The catheter was pulled out. We moved knee to let gene distribute in whole joint cavity. The body surface unipolar electrode array was wrapped on the knee, and a tape was added on the device to assure all electrodes 12, 14 were tightly contacted on the knee. Both negative and positive electrodes were connected to the pulse generator 36. A burst-electric pulse protocol with 5 ms pulse duration, 15 ms pulse interval, 10 pulses in each burst and 2 min interburst interval was applied. The electric field strength was 1 volt/cm. The knee was treated for 30 minutes.

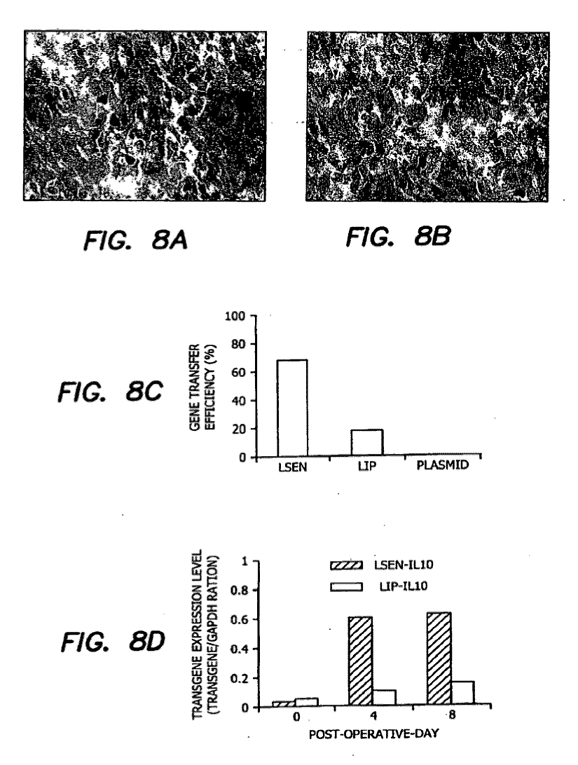

Four days after the treatment, the rabbit was sacrificed and the knee was removed. The transgene expression in articular cartilage of knee induced by LSEN-assisted IL-10 gene transfer was observed by in situ hybridization. As shown in the microphotograph of FIG. 8a, the transfection efficiency was 65±6%. As shown in the microphotograph of FIG. 8b, in another group of rabbits, the knee was treated with liposome-complexed IL-10 gene without LSEN, otherwise the procedure was the same, the gene transfer efficiency was only 13±3% as shown in the comparison graph of FIG. 8c. In knees treated with plasmid IL-10 gene only without LSEN, no any transfected cell was found. The transgene expression level determined by its ratio to the housekeeping gene GAPDH was increased 80 fold at post-operative day 4 and 8 as shown in the graph of FIG. 8d. These find provided the direct evidence that the high efficiency of LSEN-assisted gene transfer is the highest among all available viral and non-viral mediated gene transfer techniques.

In conclusion, the illustrated embodiments of the invention not only establish a method and apparatus for low strength electric field network-mediated drug and biological agents delivery in skin, soft tissue, joint and bone of large animals and humans ex vivo and in vivo, but most importantly have a very high marketing value. Skin, soft tissue, joint, and bone diseases are common within every age period. The successful treatment of these diseases has always been limited by the inefficient local drug delivery or by systemic drug use which induces adverse effects. There is no any better strategy in existence to overcome these problems. This technique is safe, cost-effective and easy to develop.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A method of transfecting a drug, gene, siRNA, shRNA, peptide, protein, antibody or a biomedical therapeutic molecule or reagent into tissue in a joint, bone, soft tissue related to the joint or bone, or into soft tissue in general comprising the steps of:
distributing the drug, gene, siRNA, shRNA, peptide, protein, antibody or a biomedical therapeutic molecule or reagent throughout the tissue;
disposing at least one positive electrode into or onto the tissue;
disposing an array of negative electrodes in proximity to the whole of the tissue to be transfected; and
applying a pulsed, low strength, network electrical field (LSEN) to whole of the tissue to be transfected.

2. The method of claim 1 where disposing the array of negative electrodes in proximity to the whole of the tissue to be transfected comprises disposing a plurality of negative electrodes into low resistance electrical contact with skin overlying the tissue.

3. The method of claim 2 where disposing the plurality of negative electrodes into low resistance electrical contact with skin overlying the tissue comprises placing the plurality of negative electrodes into tight mechanical contact with the skin.

4. The method of claim 2 where disposing a plurality of negative electrodes into low resistance electrical contact with skin overlying the tissue comprises disposing a conducting gel between the skin and the plurality of electrodes.

5. The method of claim 3 where placing the plurality of negative electrodes into tight mechanical contact with the skin comprises mechanically pressing and maintaining pressure between the plurality of negative electrodes and skin by applying folding clips and/or bands around the array and skin.

6. The method of claim 1 where distributing the drug, gene, siRNA, shRNA, peptide, protein, antibody or a biomedical therapeutic molecule or reagent throughout the tissue comprises:
inserting a guiding needle into a joint sac;
disposing an infusion catheter over or through the needle;
removing the guiding needle;
injecting the drug, gene, siRNA, shRNA, peptide, protein, antibody or a biomedical therapeutic molecule or reagent through the catheter; and
mobilizing the joint corresponding to the injected joint sac.

7. The method of claim 1 where disposing at least one positive electrode into the tissue comprises inserting a wire having a distal tip with a positive electrode on the distal tip into the infusion catheter.

8. The method of claim 1 where disposing an array of negative electrodes in proximity to the whole of the tissue to be transfected comprises placing a pad with the array of the negative electrodes included therein to cover the whole tissue to be treated.

9. The method of claim 1 where distributing the drug, gene, siRNA, shRNA, peptide, protein, antibody or a biomedical therapeutic molecule or reagent throughout the tissue comprises distributing the drug, gene, siRNA, shRNA, peptide, protein, antibody or a biomedical therapeutic molecule or reagent into an extremity by intravascular delivery using an intravenous pump or controller continuously while applying a pulsed, low strength, network electrical field (LSEN) to whole of the tissue to be transfected.

10. The method of claim 1 where distributing the drug, gene, siRNA, shRNA, peptide, protein, antibody or a biomedical therapeutic molecule or reagent throughout the tissue comprises distributing the drug, gene, siRNA, shRNA, peptide, protein, antibody or a biomedical therapeutic molecule or reagent into an extremity by topically applying the drug, gene, siRNA, shRNA, peptide, protein, antibody or a biomedical therapeutic molecule or reagent by means of a solution, oil, gel or drug delivery material while applying a pulsed, low strength, network electrical field (LSEN) to whole of the tissue to be transfected.

11. The method of claim 1 where distributing the drug, gene, siRNA, shRNA, peptide, protein, antibody or a biomedical therapeutic molecule or reagent throughout the tissue comprises distributing the drug, gene, siRNA, shRNA, peptide, protein, antibody or a biomedical therapeutic molecule or reagent into an extremity by topically applying the drug, gene, siRNA, shRNA, peptide, protein, antibody or a biomedical therapeutic molecule or reagent by subcutaneous injection while applying a pulsed, low strength, network electrical field (LSEN) to whole of the tissue to be transfected.

12. The method of claim 1 where distributing the drug, gene, siRNA, shRNA, peptide, protein, antibody or a biomedical therapeutic molecule or reagent throughout the tissue comprises distributing the drug, gene, siRNA, shRNA, peptide, protein, antibody or a biomedical therapeutic molecule or reagent into an extremity by topically applying the drug, gene, siRNA, shRNA, peptide, protein, antibody or a biomedical therapeutic molecule or reagent by application to a body surface including skin and soft tissue using tape, gel or bandages to fix the array of negative electrodes, while applying a pulsed, low strength, network electrical field (LSEN) to whole of the tissue to be transfected.

13. The method of claim 1 where distributing the drug, gene, siRNA, shRNA, peptide, protein, antibody or a biomedical therapeutic molecule or reagent throughout the tissue comprises distributing the drug, gene, siRNA, shRNA, peptide, protein, antibody or a biomedical therapeutic molecule or reagent into an extremity by topically applying the drug, gene, siRNA, shRNA, peptide, protein, antibody or a biomedical therapeutic molecule or reagent by intravascular delivery, while applying a pulsed, low strength, network electrical field (LSEN) to whole of the tissue to be transfected, the means further comprising an array of positive electrodes, where the array of positive electrodes and the array of negative electrodes are applied to a proximate body surface if the tumor is superficial, or where the array of negative electrodes are applied on one side of the tumor and the array of positive electrodes on the another side of the tumor if the tumor is on the extremity or limb, so that the fringing electric fields pass through the tumor by using an adhesion material, tape, gel or bandage to fix the electrode arrays.

14. An apparatus for transfecting a drug, gene, siRNA, shRNA, peptide, protein, antibody or a biomedical therapeutic molecule or reagent into tissue in a joint, bone, soft tissue related to the joint or bone, or into soft tissue in general comprising:
means for distributing the drug, gene, siRNA, shRNA, peptide, protein, antibody or a biomedical therapeutic molecule or reagent throughout the tissue;
at least one positive electrode inserted into or disposed on the tissue;
an array of negative electrodes disposed in proximity to the whole of the tissue to be transfected; and
a pulsed, low strength, network electrical field (LSEN) generator to apply LSEN to whole of the tissue to be transfected.

15. The apparatus of claim 14 where the array of negative electrodes disposed in proximity to the whole of the tissue to be transfected comprises a plurality of negative electrodes disposed into low resistance electrical contact with skin overlying the tissue.

16. The apparatus of claim 15 where the plurality of negative electrodes disposed into low resistance electrical contact with skin overlying the tissue comprises means for placing the plurality of negative electrodes into tight mechanical contact with the skin.

17. The apparatus of claim 15 where a plurality of negative electrodes disposed into low resistance electrical contact with skin overlying the tissue comprises a conducting gel between the skin and the plurality of electrodes.

18. The apparatus of claim 16 where the plurality of negative electrodes placed into tight mechanical contact with the skin comprises means for mechanically pressing and maintaining pressure between the plurality of negative electrodes and skin, including folding clips and/or bands around the array and skin.

19. The apparatus of claim 14 where the means for distributing the drug, gene, siRNA, shRNA, peptide, protein, antibody or a biomedical therapeutic molecule or reagent throughout the tissue comprises:
a guiding needle for insertion into a joint sac; and
an infusion catheter for disposition over or through the needle for infusing the drug, gene, siRNA, shRNA, peptide, protein, antibody or a biomedical therapeutic molecule or reagent into the joint sac.

20. The apparatus of claim 14 where the at least one positive electrode inserted into or disposed on the tissue comprises a wire having a distal tip with a positive electrode on the distal tip for insertion into the infusion catheter.

21. The apparatus of claim 14 where the array of negative electrodes in proximity to the whole of the tissue to be transfected comprises a pad with the array of the negative electrodes included therein to cover the whole tissue to be treated.

22. The apparatus of claim 14 where the means for distributing the drug, gene, siRNA, shRNA, peptide, protein, antibody or a biomedical therapeutic molecule or reagent throughout the tissue comprises means for distributing the drug, gene, siRNA, shRNA, peptide, protein, antibody or a biomedical therapeutic molecule or reagent into an extremity by intravascular delivery using an intravenous pump or controller continuously while applying a pulsed, low strength, network electrical field (LSEN) to whole of the tissue to be transfected.

23. The apparatus of claim 14 where the means for distributing the drug, gene, siRNA, shRNA, peptide, protein, antibody or a biomedical therapeutic molecule or reagent throughout the tissue comprises means for distributing the drug, gene, siRNA, shRNA, peptide, protein, antibody or a biomedical therapeutic molecule or reagent into an extremity by topically applying the drug, gene, siRNA, shRNA, peptide, protein, antibody or a biomedical therapeutic molecule or reagent by means of a solution, oil, gel or drug delivery material while applying a pulsed, low strength, network electrical field (LSEN) to whole of the tissue to be transfected.

24. The apparatus of claim 14 where the means for distributing the drug, gene, siRNA, shRNA, peptide, protein, antibody or a biomedical therapeutic molecule or reagent throughout the tissue comprises means for distributing the drug, gene, siRNA, shRNA, peptide, protein, antibody or a biomedical therapeutic molecule or reagent into an extremity by topically applying the drug, gene, siRNA, shRNA, peptide, protein, antibody or a biomedical therapeutic molecule or reagent by subcutaneous injection while applying a pulsed, low strength, network electrical field (LSEN) to whole of the tissue to be transfected.

25. The apparatus of claim 14 where the means for distributing the drug, gene, siRNA, shRNA, peptide, protein, antibody or a biomedical therapeutic molecule or reagent throughout the tissue comprises means for distributing the drug, gene, siRNA, shRNA, peptide, protein, antibody or a biomedical therapeutic molecule or reagent into an extremity by topically applying the drug, gene, siRNA, shRNA, peptide, protein, antibody or a biomedical therapeutic molecule or reagent by application to a body surface including skin and soft tissue using tape, gel or bandages to fix the array of negative electrodes, while applying a pulsed, low strength, network electrical field (LSEN) to whole of the tissue to be transfected.

26. The apparatus of claim 14 where the means for distributing the drug, gene, siRNA, shRNA, peptide, protein, antibody or a biomedical therapeutic molecule or reagent throughout the tissue comprises means for distributing the drug, gene, siRNA, shRNA, peptide, protein, antibody or a biomedical therapeutic molecule or reagent into an extremity by topically applying the drug, gene, siRNA, shRNA, peptide, protein, antibody or a biomedical therapeutic molecule or reagent by intravascular delivery, while applying a pulsed, low strength, network electrical field (LSEN) to whole of the tissue to be transfected, the means further comprising an array of positive electrodes, where the array of positive electrodes and the array of negative electrodes are applied to a proximate body surface if the tumor is superficial, or where the array of negative electrodes are applied on one side of the tumor and the array of positive electrodes on the another side of the tumor if the tumor is on the extremity or limb, so that the fringing electric fields pass through the tumor by using an adhesion material, tape, gel or bandage to fix the electrode arrays.

27. The method of claim 1 where distributing the drug, gene, siRNA, shRNA, peptide, protein, antibody or a biomedical therapeutic molecule or reagent throughout the tissue comprises distributing at least one of the members of the group consisting of:
1) leukocyte markers, such as CD2, CD3, CD4, CD5, CD6, CD7, CD8, CD11a,b,c, CD13, CD14, CD18, CD19, CD20, CD22, CD23, CD25, CD27 and its ligand, CD28 and its ligands B7.1, B7.2, B7.3, CD29 and its ligand, CD30 and its ligand, CD40 and its ligand gp39, CD44, CD45 and isoforms, Cdw52 (Campath antigen), CD56, CD58, CD69, CD72, CD80, CD86, CTLA-4, CTLA4Ig, LFA-1 and TCR or a mutant thereof, including LEA29Y; adhesion molecule inhibitors, such as LFA-1 antagonists, ICAM-1 or -3 antagonists, VCAM-4 antagonists or VLA-4 antagonists; or a chemotherapeutic agent;
2) histocompatibility antigens, such as MHC class I or II, Lewis Y antigens, Slex, Sley, Slea, and Selb;
3) adhesion molecules, including integrins, such as VLA-1, VLA-2, VLA-3, VLA-4, VLA-5, VLA-6, LFA-1, Mac-1, αVβ3, and p150, 95; 4) the selectins, such as L-selectin, E-selectin, and P-selectin and their counterreceptors VCAM-1, ICAM-1, ICAM-2, and LFA-3;
5) interleukins, such as IL-1, IL-2, IL-3, IL-4, IL-5, IL-6, IL-7, IL-8, IL-9, IL-10, IL-11, IL-12, IL-13, IL-14, and IL-15;
6) interleukin receptors, such as IL-1R, IL-2R, IL-3R, IL-4R, IL-5R, IL-6R, IL-7R, IL-8R, IL-9R, IL-10R, IL-11R, IL-12R, IL-13R, IL-14R and IL-15R;
7) chemokines, such as PF4, RANTES, MIP1a, MCP1, IP-10, ENA-78, NAP-2, Gro-α, Gro-β, and IL-8;
8) growth factors, such as TNFα, TGFβ, TSH, VEGF/VPF, PTHrP, EGF family, FGF, PDGF family, endothelin, Fibrosin (F.sub.sF.sub.−1), Laminin, and gastrin releasing peptide (GRP);
9) growth factor receptors, such as TNFαR, RGFβR, TSHR, VEGFR/VPFR, FGFR, EGFR, PTHrPR, PDGFR family, EPO-R, GCSF-R and other hematopoietic receptors;
10) interferon receptors, such as IFN-αR, IFN-βR, and IFN.sub.YR;
11) Igs and their receptors, such as IGE, FceRI, and FceRII;
12) tumor antigens, such as her2-neu, mucin, CEA and endosialin;
13) allergens, such as house dust mite antigen, IoI p1 (grass) antigens, and urushiol;
14) viral proteins, such as CMV glycoproteins B, H, and gCIII, HIV-1 envelope glycoproteins, RSV envelope glycoproteins, HSV envelope glycoproteins, EBV envelope glycoproteins, VZV, envelope glycoproteins, HPV envelope glycoproteins, Hepatitis family surface antigens;
15) toxins, such as pseudomonas endotoxin and osteopontin/uropontin, snake venom, spider venom, or bee venom;
16) blood factors, such as complement C3b, complement C5a, complement C5b-9, Rh factor, fibrinogen, fibrin, or myelin associated growth inhibitor;
17) enzymes, such as cholesterol ester transfer protein, membrane bound matrix metalloproteases, and glutamic acid decarboxylase (GAD);
18) miscellaneous antigens including ganglioside GD3, ganglioside GM2, LMP1, LMP2, eosinophil major basic protein, PTHrp, eosinophil cationic protein, pANCA, Amadori protein, Type IV collagen, glycated lipids, nu-interferon, A7, P-glycoprotein and Fas (AFO-1) and oxidized-LDL
19) calcineurin inhibitor, such as cyclosporin A or FK 506;
20) mTOR inhibitor, such as rapamycin, 40-O-(2-hydroxyethyl)-rapamycin, CCI779, ABT578 or AP23573;
21) an ascomycin having immunosuppressive properties, such as ABT-281, ASM981;
22) corticosteroids; cyclophosphamide; azathioprene; methotrexate; leflunomide; mizoribine; mycophenolic acid; mycophenolate mofetil; 15 deoxyspergualine or an immunosuppressive homologue, analogue or derivative thereof;
23) apoptosis genes; or
24) any combination of the members of the group.

28. The method of claim 27 where distributing at least one of the genes, proteins or antibodies consisting of the members of the group comprises administering the member as the sole active ingredient or in conjunction with or as an adjuvant to other drugs, immunosuppressive or immunomodulating agents or other anti-inflammatory agents, for the treatment or prevention of allo- or xenograft acute or chronic rejection or inflammatory or autoimmune disorders, or as a chemotherapeutic agent or as a malignant cell anti-proliferative agent, where the chemotherapeutic agent comprises a member of the group consisting of:
i. an aromatase inhibitor,
ii. a microtubule active agent, an alkylating agent, an antineoplastic antimetabolite or a platin compound,
iii. a compound targeting/decreasing a protein or lipid kinase activity or a protein or lipid phosphatase activity, a further anti-angiogenic compound or a compound which induces cell differentiation processes,
iv. a bradykinin 1 receptor or an angiotensin II antagonist,
v. a cyclooxygenase inhibitor, a bisphosphonate, a histone deacetylase inhibitor, a heparanase inhibitor (prevents heparan sulphate degradation), such as PI-88, a biological response modifier, preferably a lymphokine or interferons, such as interferon quadrature., an ubiquitination inhibitor, or an inhibitor which blocks anti-apoptotic pathways,
vi. an inhibitor of Ras oncogenic isoforms, such as H-Ras, K-Ras or N-Ras, or a farnesyl transferase inhibitor, such as L-744,832 or DK8G557,
vii. a telomerase inhibitor, such as telomestatin,
viii. a protease inhibitor, a matrix metalloproteinase inhibitor, a methionine aminopeptidase inhibitor, such as bengamide or a derivative thereof, or a proteosome inhibitor, such as PS-341, or
ix. a mTOR inhibitor; or
x. any combination of members of the group.

29. The method of claim 1 where distributing the drug, gene, siRNA, shRNA, peptide, protein, antibody or a biomedical therapeutic molecule or reagent throughout the tissue comprises distributing an inhibitor, enhancer, agonist, antagonist, regulator, modulator, modifier, or monitor of the drug, gene, siRNA, shRNA, peptide, protein, antibody or a biomedical therapeutic molecule or reagent.
